(19) United States
(12) Patent Application Publication
(10) Pub. No.: US 2024/0008981 A1
(43) Pub. Date: Jan. 11, 2024

(54) IMPLANTABLE MEDICAL DEVICE WITH VISUAL ORIENTATION INDICATOR

(71) Applicant: BOSTON SCIENTIFIC SCIMED, INC., MAPLE GROVE, MN (US)

(72) Inventors: Tim O'Connor, Galway (IE); Neil O'Connor, Galway (IE); Declan Loughnane, Galway (IE); Joseph Murphy, Galway (IE)

(73) Assignee: BOSTON SCIENTIFIC SCIMED, INC., MAPLE GROVE, MN (US)

(21) Appl. No.: 18/219,302

(22) Filed: Jul. 7, 2023

Related U.S. Application Data
(60) Provisional application No. 63/359,595, filed on Jul. 8, 2022.

(51) Int. Cl. A61F 2/24

(52) U.S. Cl. CPC A61F 2/2442; A61F 2/243; A61F 2220/0075; A61F 2250/0098; A61F 2230/0043

(57) ABSTRACT

An implantable medical device adapted to be implanted at an implantation site may be capable of being implanted in more than one rotational orientation at the implantation site. The implantable medical device includes an expandable body that is adapted to expand from a collapsed configuration for delivery to an expanded configuration for deployment, and a radiopaque indicator that is disposed relative to the expandable body. The radiopaque indicator is adapted to be have a first appearance if viewed in a first rotational orientation and a second, different appearance if viewed in a second rotational orientation that is different from the first rotational orientation. As an example, the implantable medical device may be a catheter-delivered replacement aortic valve.

### CROSS REFERENCE TO RELATED APPLICATIONS

This application claims the benefit of priority of U.S. Provisional Application No. 63/359,595 filed Jul. 8, 2022, the entire disclosure of which is hereby incorporated by reference.

### TECHNICAL FIELD

The present disclosure pertains to medical devices, and methods for manufacturing and using medical devices. More particularly, the disclosure is directed to implantable medical devices having a visual indicator showing appropriate orientation during and after implantation.

### BACKGROUND

A wide variety of medical devices have been developed for medical use, for example, for use in accessing body cavities and interacting with fluids and structures in body cavities. Some of these devices may include guidewires, catheters, pumps, motors, controllers, filters, grinders, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### SUMMARY

This disclosure provides design, material, manufacturing method, and use alternatives for medical devices. An example may be found in an implantable medical device that is adapted to be implanted at an implantation site and is capable of being implanted in more than one rotational orientation. The implantable medical device includes an expandable body that is adapted to expand from a collapsed configuration for delivery to an expanded configuration for deployment and a radiopaque indicator disposed relative to the expandable body, the radiopaque indicator adapted to have a first appearance if viewed in a first rotational orientation and a second, different appearance if viewed in a second rotational orientation that is different from the first rotational orientation.

Alternatively or additionally, the radiopaque indicator may be visible under fluoroscopy, and thus able to indicate orientation of the implantable medical device, while the implantable medical device remains within a delivery device being used to deliver the implantable medical device.

Alternatively or additionally, the radiopaque indicator may be visible under fluoroscopy, and thus able to indicate orientation of the implantable medical device, after the implantable medical device has been at least partially deployed from the delivery device.

Alternatively or additionally, the radiopaque indicator may include a shape emulating an alphanumeric character.

Alternatively or additionally, the radiopaque indicator may include a shape emulating an alphanumeric character when viewed from a front of the shape and may appear as an asymmetric mirror image of the alphanumeric character when viewed from a back of the shape.

Alternatively or additionally, the radiopaque indicator may include a rectilinear cross-sectional profile.

Alternatively or additionally, the radiopaque indicator may include tantalum.

Alternatively or additionally, the implantable medical device may include a replacement heart valve.

Alternatively or additionally, the replacement heart valve may include a first layer of valve flap material and a second layer of valve flap material, and the radiopaque indicator may be secured to the first layer and/or the second layer of valve flap material.

Alternatively or additionally, the radiopaque indicator may be sutured to the first layer and/or the second layer of valve flap material.

Alternatively or additionally, the radiopaque indicator may be disposed between the first layer of valve flap material and the second layer of valve flap material.

Another example may be found in a replacement cardiac valve that is adapted to be implanted within a native cardiac valve annulus and is capable of being implanted in more than one rotational orientation. The replacement cardiac valve includes an expandable body that is adapted to expand from a collapsed configuration for delivery to an expanded configuration for deployment, the expandable body including a plurality of replacement valve commissure posts. A valve material is secured relative to the plurality of replacement valve commissure posts, with the valve material forming a valve cusp between each of the plurality of replacement valve commissure posts. A radiopaque indicator is disposed relative to one of the plurality of replacement valve commissure posts, the radiopaque indicator adapted to provide an indication of a rotational orientation of the replacement cardiac valve relative to the native cardiac valve annulus.

Alternatively or additionally, the native cardiac valve may include an aortic valve, the replacement cardiac valve may include a replacement aortic valve, and the radiopaque indicator may be adapted to provide an indication of a relative position of each of the plurality of commissure posts relative to coronary arteries proximate the native aortic valve annulus.

Alternatively or additionally, the radiopaque indicator may be secured relative to the valve material.

Alternatively or additionally, the radiopaque indicator may be sutured to the valve material and/or one of the replacement valve commissure posts.

Alternatively or additionally, the radiopaque indicator may be positioned such that the radiopaque indicator is visible under fluoroscopy before and during deployment of the replacement cardiac valve.

Alternatively or additionally, the radiopaque indicator may include a shape emulating an alphanumeric character when viewed from a position in front of the shape and may appear as an asymmetric a mirror image of the alphanumeric character when viewed from a position behind the shape.

Alternatively or additionally, the shape may include one of a “C” shape, an “E” shape, an “F” shape, a “J” shape, a “K” shape, an “L” shape, a “P” shape, an “R” shape, or a “Z” shape.

Another example may be found in a replacement aortic valve that is adapted to be implanted within a native aortic valve having a plurality of native commissures and is capable of being implanted in more than one rotational orientation. The replacement aortic valve includes an expandable body that is adapted to expand from a collapsed configuration for delivery to an expanded configuration for deployment, the expandable body including a plurality of replacement valve commissure posts. A first layer of a bovine valve material is disposed on a first side of the plurality of replacement valve commissure posts. A second layer of a bovine valve material is disposed on a second side of the plurality of replacement valve commissure posts. A radiopaque indicator is disposed between the first layer of bovine material and the second layer of bovine material, the radiopaque indicator adapted to provide an indication of a rotational orientation of the replacement aortic valve relative to the native aortic valve.

Alternatively or additionally, the radiopaque indicator may include an “L” shape formed of tantalum.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1A is a view of an illustrative replacement aortic valve expandable frame;

FIG. 1B is a view of an illustrative replacement aortic valve including the expandable frame of FIG. 1A;

FIG. 2A is a schematic view of a replacement aortic valve misaligned with respect to the native aortic valve;

FIG. 2B is a schematic view of a replacement aortic valve that is appropriately aligned with the native aortic valve;

FIG. 3 is a schematic view of a portion of an illustrative replacement aortic valve including tissue layers, a radiopaque indicator and a portion of the expandable frame

FIG. 4 is a fluoroscopic image of an illustrative replacement aortic valve within its delivery device, tracked to position relative to a schematically illustrated native aortic valve annulus;

FIG. 5 is a fluoroscopic image of the illustrative replacement aortic valve of FIG. 4, shown after phase one of releasing the replacement aortic valve from its delivery device with the replacement aortic valve properly aligned with the native aortic valve annulus;

FIG. 6 is a fluoroscopic image of the illustrative replacement aortic valve of FIG. 4, shown after phase two of releasing the replacement aortic valve from its delivery device with the replacement aortic valve properly aligned with the native aortic valve annulus; and

FIG. 7 is a fluoroscopic image of the illustrative replacement aortic valve of FIG. 4, shown after phase one of releasing the replacement aortic valve from its delivery device with the replacement aortic valve not properly aligned with the native aortic valve annulus.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

A number of implantable medical devices are implanted at a variety of different implantation sites within a patient. In some cases, some implantable medical devices are capable of being implanted in more than one possible orientation at a particular implantation site. For example, some implantable medical devices, by basis of their configuration or overall shape, may be capable of being implanted in more than one rotational orientation. The implantable medical device may be implanted such that a particular reference point on the implantable medical device, for example, may be facing any particular rotational point as defined along a 360 degree circle. The reference point may be facing a direction of 45 degrees, or perhaps 310 degrees, or any of a variety of different directions. For some implantable medical devices, the rotational orientation may not matter. For some implantable medical devices, the rotational orientation may be important.

For ease of illustration, the disclosure is directed to the implantable medical device being a replacement cardiac valve, such as a replacement aortic valve, that is deliverable in a trans-catheter manner. However, the disclosure is intended to not be so limited, as the replacement aortic valve described herein is merely illustrative.

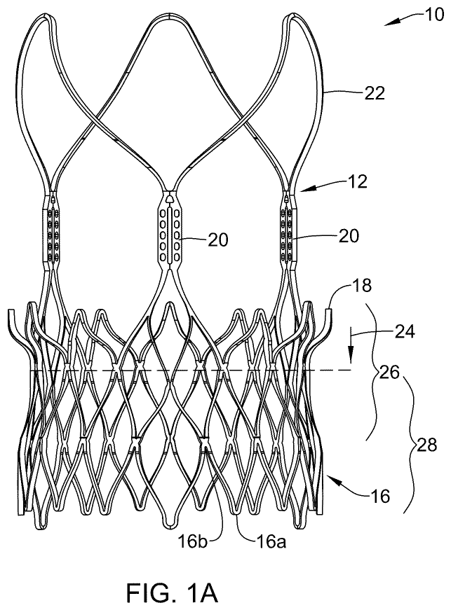

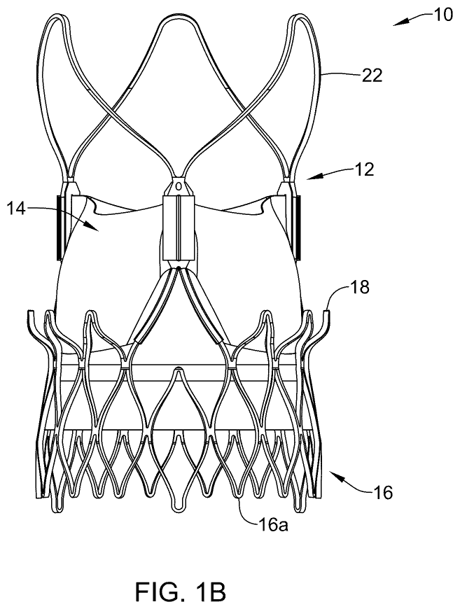

FIG. 1A and FIG. 1B are side views of an illustrative replacement cardiac valve 10. The replacement cardiac valve 10 may be a replacement aortic valve, a replacement mitral valve, a replacement pulmonary valve or a replacement tricuspid valve, for example. In some cases, the replacement cardiac valve 10 may include biological tissue such as porcine or bovine pericardium and/or natural cardiac valve leaflets such as natural porcine cardiac valve leaflets. In some cases, the natural cardiac valve leaflets may be attached to a portion of natural cardiac wall tissue. The biological material may be fixed, for example, using glutaraldehyde.

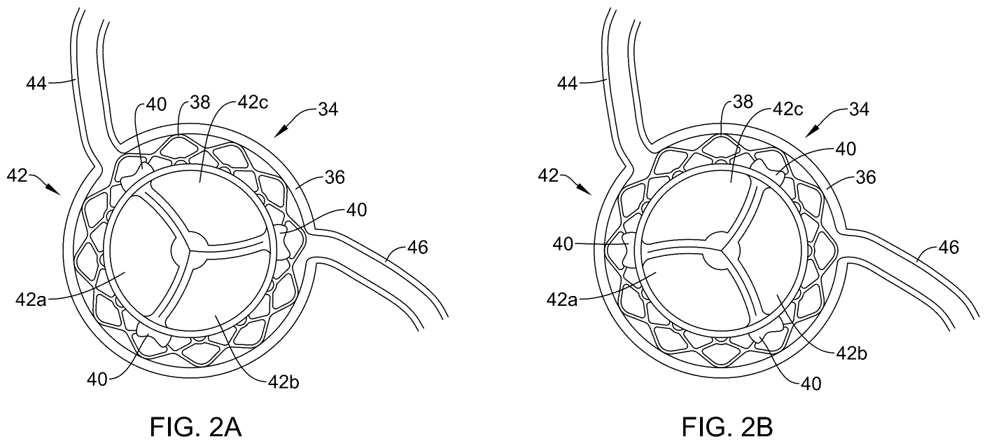

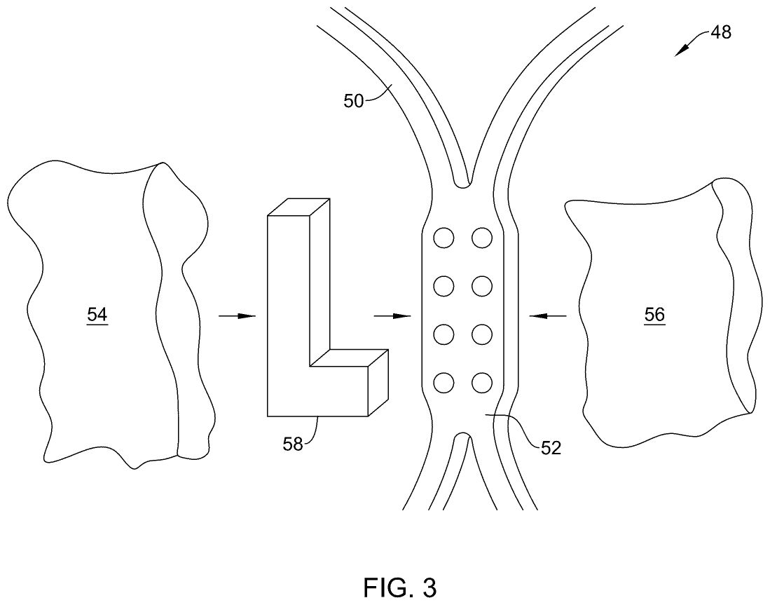

The replacement cardiac valve 10 includes an expandable frame 12 that may be compressible to a radially compressed, or collapsed, configuration for delivery using a delivery catheter, and may be expandable to an expanded configuration (as shown) during implantation. The replacement cardiac valve 10 may include a plurality of leaflets defining a valve 14 (as seen in FIG. 1), the position of which is depicted schematically by the bounding phantom lines. The leaflets defining the valve 14 are visible in FIGS. 2A and 2B (including leaflets 42a, 42b and 42c), for example.

In some cases, the expandable frame may include a lower tubular or crown portion 16, an upper crown portion 18, a plurality of upstanding commissural posts 20, and a plurality of stabilization arches 22. In use, the lower portion 16 of the expandable frame 12 may be adapted to be deployed after the other regions of the expandable frame 12. For example, the arches 22, the supports 20 and the upper crown 18 may be deployed at least partly before the lower portion 16 (in that order, or in reverse order, or in a different order). At the very least, once the upper crown 18 has been at least partly deployed, the expandable frame 12 may be urged and/or displaced in the direction of arrow 24 to seat the upper crown 18 against native leaflets at the implantation site. Deploying the lower portion 16 last fixes the expandable frame 12 in its final position.

The lower portion 16, and optionally a portion of the upper crown 18, may be formed by a lattice structure of the stent. The lattice structure may define cells or apertures, for example, generally diamond-shaped apertures. In some cases, the native leaflets may generally overlap a portion 26 of the expandable frame 12. The native valve annulus may overlap a portion 28 of the expandable frame.

The expandable frame 12 may optionally be of a self-expanding type that is compressible to the compressed configuration for loading into a delivery catheter for delivery to the site of implantation. In use, by removal of the constraining effect of a sheath holding the expandable frame 12 in the compressed configuration, the expandable frame 12 self-expands to or towards the operative configuration. A self-expanding stent may, for example, be of shape-memory material, for example, shape-memory metal alloy, for example, nitinol. Alternatively, the expandable frame 12 may be configured to be expanded by application of a foreshortening force from the delivery catheter and/or by application of expanding force from the delivery catheter, such as by using an expansion balloon. These are just examples.

In some cases, the relative rotational orientation of the replacement cardiac valve 10 may be important, particularly when the replacement cardiac valve 10 is a replacement aortic valve, intended for implantation within a native aortic annulus. In some cases, portions of the replacement cardiac valve 10 may potentially interfere with subsequent access to the coronary arteries. In some patients, particularly younger patients, there may be a subsequent need to implant a new replacement aortic valve some years after the initial implantation. In some patients, there may be a need, either immediate or down the road, to access one or more of the coronary arteries in order to perform angioplasty or rotational atherectomy, for example. There may be a need to access one or more of the coronary arteries in order to implant one or more stents. If the initially implanted replacement aortic valve interferes with access to the coronary arteries, this can be problematic.

FIG. 2A shows a schematic view of a replacement aortic valve 34 that has been implanted within a native aortic annulus 36. The replacement aortic valve 34 may be considered as being an example of the replacement cardiac valve 10 shown and described in FIGS. 1A and 1B. The replacement aortic valve 34 includes an expandable frame 38 that may be similar to the expandable frame 12. The expandable frame 38 includes several commissure posts 40. In some case, valve leaflets 42, individually labeled as 42a, 42b and 42c, are secured relative to the commissure posts 40. As shown, a coronary artery 44 and a coronary artery 46 are each connected to the native aortic annulus 36.

FIG. 2A illustrates a poor rotational orientation of the replacement aortic valve 34, as it can be seen that one of the commissure posts 40 is at least partially blocking the coronary artery 44 and another one of the commissure posts 40 is at least partially blocking the coronary artery 46. While both coronary arteries 44 and 46 are shown as being partially blocked, in some cases the particular location of the coronary arteries 44 and 46 in some patients may mean that a poor rotational orientation of the replacement aortic valve 34 may result in only one of the coronary arteries 44 and 46 being at least partially blocked.

FIG. 2B illustrates an optimal rotation orientation of the replacement aortic valve 34, as it can be seen that none of the commissure posts 40 are blocking the coronary artery 44 and none of the commissure posts 40 are blocking the coronary artery 46. As a result of this rotational orientation, the replacement aortic valve 34 will not cause problems, or at least should cause fewer problems, if the need arises in the future to implant a second replacement aortic valve. This also means that the coronary arteries 44 and 46 are easily reachable for any ensuing procedures within the coronary arteries 44 and 46, such as but not limited to angioplasty, rotational atherectomy or stent implantation.

FIG. 3 is a schematic view of a portion of an illustrative replacement cardiac valve 48. The illustrative replacement cardiac valve 48 may be considered as being an example of the replacement cardiac valve 10 as shown and described in FIGS. 1A and 1, and/or the replacement aortic valve 34 as shown and described in FIGS. 2A and 2B. The replacement cardiac valve 48 includes an expandable frame 50 that includes a number of commissure posts 52 (only one is shown). In some cases, the replacement cardiac valve 48 includes a first tissue layer 54 and a second tissue layer 56 that are disposed on either side of the commissure posts 52, thereby sandwiching the commissure posts 52 between the first tissue layer 54 and the second tissue layer 56. In some cases, the first tissue layer 54 and/or the second tissue layer 56 may be considered as part of the tissue forming the valve 14.

A radiopaque indicator 58 may be disposed between the first tissue layer 54 and the second tissue layer 56. In some cases, the radiopaque indicator 58 may be disposed between the first tissue layer 54 and the commissure post 52, as shown. In some instances, the radiopaque indicator 58 may instead be disposed between the commissure post 52 and the second tissue layer 56. In some cases, the radiopaque indicator 58 may be secured to the commissure post 52. In some cases, the radiopaque indicator 58 may be secured to one or both of the first tissue layer 54 and the second tissue layer 56. The radiopaque indicator 58 may be adhesively secured in place. The radiopaque indicator 58 may be sutured into position. In some cases, the radiopaque indicator 58 may be secured in position relative to the commissure post 52 such that the radiopaque indicator 58 is visible under fluoroscopy while the replacement cardiac valve 48 is in a compressed configuration for delivery. In some cases, the radiopaque indicator 58 may be secured in position relative to the commissure post 52 such that the radiopaque indicator 58 is visible under fluoroscopy after the replacement cardiac valve 48 has been deployed, or even during deployment of the replacement cardiac valve 48.

The radiopaque indicator 58 may be formed of any suitable radiopaque material that is sufficiently visible under fluoroscopy, for example. In some cases, the radiopaque indicator 58 may be formed of tantalum. In some cases, the radiopaque indicator 58 may be formed of a platinum-iridium mix or alloy, gold, tungsten, bismuth or barium. The radiopaque indicator 58 may be formed by cutting and shaping a length of a wire of the suitable material. As an illustrative but non-limiting example, the radiopaque indicator 58 may be formed from a length of tantalum wire that has a 0.020 inches diameter. In some cases, the radiopaque indicator 58 may be formed of a wire having a square or otherwise rectilinear cross-sectional profile, in order to maximize radiopacity (and thus maximize visibility during fluoroscopy).

In some cases, the radiopaque indicator 58 may have a first appearance when viewed in a first rotational orientation and a second, different appearance when viewed in a second rotational orientation that is different from the first rotational orientation. As an example, the radiopaque indicator 58 may have a shape that emulates an alphanumeric character when viewed from a position in front of the shape and appears as an asymmetric mirror image of the alphanumeric character when viewed from a position behind the shape. In some cases, the radiopaque indicator 58 may have a shape such as one of a “C” shape, an “E” shape, an “F” shape, a “J” shape, a “K” shape, an “L” shape, a “P” shape, an “R” shape, or a “Z” shape. These are just examples, as other shapes are also contemplated.

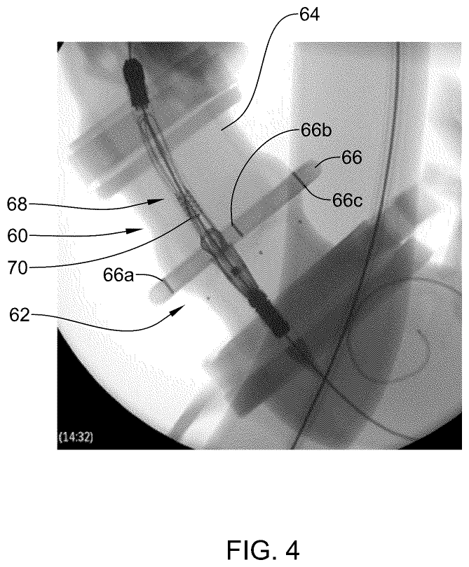

FIGS. 4 through 6 are fluoroscopic images showing an illustrative replacement aortic valve 60 that is being delivered to a treatment site via a delivery catheter 62. In these images, the treatment site is an artificially-created aortic valve implantation site 64, constructed out of polymeric pipe to be fluoroscopically translucent. The artificially-created aortic valve implantation site 64 includes an element 66, bearing three markers 66a, 66b and 66c, emulating an aortic annulus and the relative locations of the LCC (left coronary cusp), the RCC (right coronary cusp) and the NCC (non-coronary cusp). The LCC corresponds to the valve leaflet proximate the left coronary artery and the RCC corresponds to the valve leaflet proximate the right coronary artery. One of the coronary arteries 44 and 46 shown in FIGS. 2A and 2B corresponds to the left coronary artery while the other of the coronary arteries 44 and 46 corresponds to the right coronary artery.

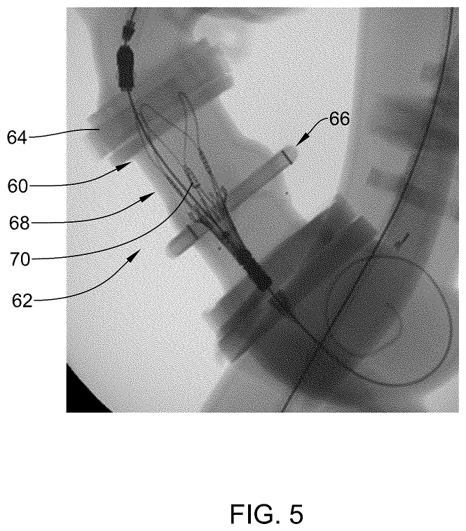

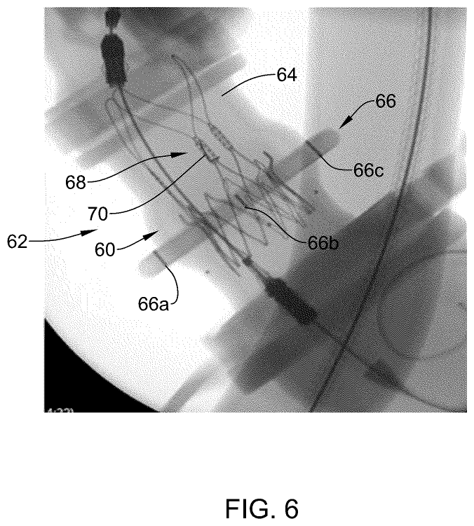

As seen in FIG. 4, the replacement aortic valve 60 has tracked to position, with a commissure post 68 bearing a radiopaque indicator 70 at the middle posterior position. In this example, the radiopaque indicator 70 has the shape of an “L”. Because the “L” appears correctly as an “L”, this means that the aortic valve 60 is currently in an appropriate rotational orientation. Moving to FIG. 5, the replacement aortic valve 60 has moved to a post release phase one position, with the commissure post 68 bearing the radiopaque indicator 70 at the posterior position. Because the “L” still appears correctly as an “L”, this means that the aortic valve 60 is still in an appropriate rotational orientation. Moving to FIG. 6, the replacement aortic valve 60 has moved to a post release phase two position, with the commissure post 68 bearing the radiopaque indicator 70 at the posterior position. Because the “L” still appears correctly as an “L”, this means that the aortic valve 60 is still in an appropriate rotational orientation.

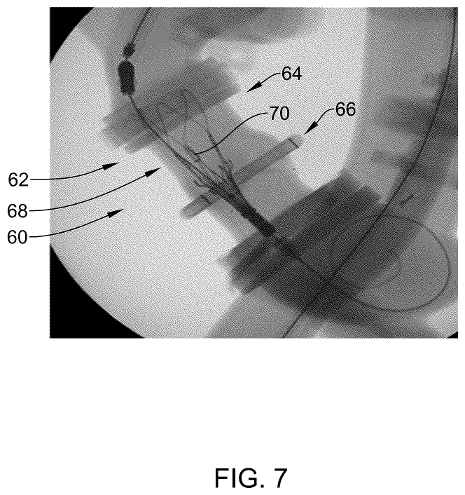

FIG. 7 provides an example in which the replacement aortic valve 60 is in a poor rotational orientation. In FIG. 7, the replacement aortic valve 60 is at a delivery stage similar to that shown in FIG. 5. However, in FIG. 7, the radiopaque indicator 70 is not appearing as an “L” shape, but rather as the mirror image. This means that the replacement aortic valve 60 is rotated with respect to the position shown in FIG. 5. For example, the replacement aortic valve 60 as shown in FIG. 7 may be rotated about 180 degrees from its position as shown in FIG. 5.

The devices described herein, as well as various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An implantable medical device adapted to be implanted at an implantation site, the implantable medical device capable of being implanted in more than one rotational orientation, the implantable medical device comprising:
an expandable body that is adapted to expand from a collapsed configuration for delivery to an expanded configuration for deployment; and
a radiopaque indicator disposed relative to the expandable body, the radiopaque indicator adapted to have a first appearance if viewed in a first rotational orientation and a second, different appearance if viewed in a second rotational orientation that is different from the first rotational orientation.

2. The implantable medical device of claim 1, wherein the radiopaque indicator is visible under fluoroscopy, and thus able to indicate orientation of the implantable medical device, while the implantable medical device remains within a delivery device being used to deliver the implantable medical device.

3. The implantable medical device of claim 1, wherein the radiopaque indicator is visible under fluoroscopy, and thus able to indicate orientation of the implantable medical device, after the implantable medical device has been at least partially deployed from the delivery device.

4. The implantable medical device of claim 1, wherein the radiopaque indicator comprises a shape emulating an alphanumeric character.

5. The implantable medical device of claim 4, wherein the radiopaque indicator comprises a shape emulating an alphanumeric character when viewed from a front of the shape and appears as an asymmetric mirror image of the alphanumeric character when viewed from a back of the shape.

6. The implantable medical device of claim 4, wherein the radiopaque indicator comprises a rectilinear cross-sectional profile.

7. The implantable medical device of claim 1, wherein the radiopaque indicator comprises tantalum.

8. The implantable medical device of claim 1, comprising a replacement heart valve.

9. The implantable medical device of claim 8, wherein the replacement heart valve comprises a first layer of valve flap material and a second layer of valve flap material, and the radiopaque indicator is secured to the first layer and/or the second layer of valve flap material.

10. The implantable medical device of claim 9, wherein the radiopaque indicator is sutured to the first layer and/or the second layer of valve flap material.

11. The implantable medical device of claim 9, wherein the radiopaque indicator is disposed between the first layer of valve flap material and the second layer of valve flap material.

12. A replacement cardiac valve adapted to be implanted within a native cardiac valve annulus, the replacement cardiac valve capable of being implanted in more than one rotational orientation, the replacement cardiac valve comprising:
an expandable body that is adapted to expand from a collapsed configuration for delivery to an expanded configuration for deployment, the expandable body including a plurality of replacement valve commissure posts;
a valve material secured relative to the plurality of replacement valve commissure posts, with the valve material forming a valve cusp between each of the plurality of replacement valve commissure posts; and
a radiopaque indicator disposed relative to one of the plurality of replacement valve commissure posts, the radiopaque indicator adapted to provide an indication of a rotational orientation of the replacement cardiac valve relative to the native cardiac valve annulus.

13. The replacement cardiac valve of claim 12, wherein:
the native cardiac valve comprises an aortic valve;
the replacement cardiac valve comprises a replacement aortic valve; and
the radiopaque indicator is adapted to provide an indication of a relative position of each of the plurality of commissure posts relative to coronary arteries proximate the native aortic valve annulus.

14. The replacement cardiac valve of claim 12, wherein the radiopaque indicator is secured relative to the valve material.

15. The replacement cardiac valve of claim 12, wherein the radiopaque indicator is sutured to the valve material and/or one of the replacement valve commissure posts.

16. The replacement cardiac valve of claim 12, wherein the radiopaque indicator is positioned such that the radiopaque indicator is visible under fluoroscopy before and during deployment of the replacement cardiac valve.

17. The replacement cardiac valve of claim 12, wherein the radiopaque indicator comprises a shape emulating an alphanumeric character when viewed from a position in front of the shape and appears as an asymmetric mirror image of the alphanumeric character when viewed from a position behind the shape.

18. The replacement cardiac valve of claim 17, wherein the shape comprises one of a “C” shape, an “E” shape, an “F” shape, a “J” shape, a “K” shape, an “L” shape, a “P” shape, an “R” shape, or a “Z” shape.

19. A replacement aortic valve adapted to be implanted within a native aortic valve having a plurality of native commissures, the replacement aortic valve capable of being implanted in more than one rotational orientation, the replacement aortic valve comprising:
an expandable body that is adapted to expand from a collapsed configuration for delivery to an expanded configuration for deployment, the expandable body including a plurality of replacement valve commissure posts;
a first layer of a bovine valve material disposed on a first side of the plurality of replacement valve commissure posts;
a second layer of a bovine valve material disposed on a second side of the plurality of replacement valve commissure posts; and
a radiopaque indicator disposed between the first layer of bovine material and the second layer of bovine material, the radiopaque indicator adapted to provide an indication of a rotational orientation of the replacement aortic valve relative to the native aortic valve.

20. The replacement aortic valve of claim 19, wherein the radiopaque indicator comprises an “L” shape formed of tantalum.
